(19) United States
(12) Patent Application Publication
(10) Pub. No.: US 2026/0184699 A1
(43) Pub. Date: Jul. 2, 2026

(54) METHOD FOR PREPARING COMPOUND II

(71) Applicant: JIANGXI TIANYU CHEMICAL CO., LTD., Ji'an City (CN)

(72) Inventors: Dongjiang ZHAO, Ji'an City (CN); Kankan ZHOU, Ji'an City (CN); Yingzi XIAO, Ji'an City (CN); Hongxia LIU, Ji'an City (CN); Chaoqun HUANG, Ji'an City (CN); Huailin PANG, Ji'an City (CN)

(21) Appl. No.: 19/245,543

(22) Filed: Jun. 23, 2025

(30) Foreign Application Priority Data
Dec. 26, 2024 (CN) 202411940868.X

(51) Int. Cl. C07D 413/12

(52) U.S. Cl. CPC C07D 413/12

(57) ABSTRACT

Provided is a method for preparing Compound II, wherein the method includes the following steps: using Compound I as a raw material, performing a reaction under the action of an organometallic catalyst and a phase transfer catalyst, and oxidizing with hydrogen peroxide to obtain Compound II. The catalyst utilized in the present application exhibits remarkably high activity for the oxidation of sulfur ether compounds, leading to the preparation of sulfone compounds. The method for preparing Compound II significantly reduces the amount of catalyst required, effectively diminishing the consumption of non-renewable scarce strategic resources, while concurrently reducing the potential hazards to the natural environment. The novel method exhibits a substantial reduction in the amount of catalyst required, leading to a concurrent decrease in the consumption of non-renewable resources. Concurrently, it significantly mitigates potential harm to the natural environment and human health. The solvent utilized in this method is easy to be recycled and reused, and enhances overall process efficiency by reducing resource consumption, waste generation, and treatment costs, thereby rendering it more suitable for industrial production.

### CROSS-REFERENCE TO RELATED APPLICATIONS

This application claims priority to Chinese Patent Application No. 202411940868.X filed on Dec. 26, 2024, the disclosure of which is incorporated herein by reference in its entirety.

### TECHNICAL FIELD

The present application belongs to the field of pesticide technology, and specifically relates to a method for preparing Compound II.

### BACKGROUND

Compound II (the molecular structure of which is set forth below in the “SUMMARY”) is an isoxazole herbicide which is a potent inhibitor of VLCFA (very long chain fatty acid) (C20-C30) biosynthesis in plants. It is absorbed by the seedling roots and shoots of weeds after application and inhibits the growth of the seedling. It is used as a pre-emergence soil treatment for the control of annual grasses and broadleaf weeds in corn, cotton, soybeans, etc. It is particularly effective for controlling Digitaria sanguinalis (crabgrass), Setaria viridis (green foxtail), Eleusine indica (goosegrass), Echinochloa crusgalli (barnyardgrass), Leptochloa chinensis (Chinese sprangletop), Apera spica-venti (windgrass), and annual broadleaf weeds such as Amaranthus spp., Chenopodium spp., Polygonum spp., Commelina diffusa (dayflower), Gallium spurium (gallium), and Cuscuta spp.

WO2021002484 discloses a preparation method of Compound II, which is prepared by oxidation of raw material Compound I (the molecular structure of which is set forth in the “SUMMARY” hereinafter) under the catalysis of sodium tungstate dihydrate or ammonium molybdate tetrahydrate with hydrogen peroxide, involving various solvents and various catalysts. The Compound II yield can reach up to 95.9% (the purity is not clearly reported), which is relatively high.

However, tungsten and molybdenum are non-renewable scarce strategic resources. The content of them in the Earth Crust is only about 0.001%. Tungsten alloy with its high melting point and hardness is widely used in national defense military (atomic bomb, rocket), aerospace, machining, metallurgy, oil drilling, mining tools, electronic communications, construction and other fields. Countries such as the United States and Russia have established national strategic reserves of tungsten (as shown in the tungsten industry reports, of which the link is: https://doc.guandang.net/bfb8623386ec4259baf0fca5fhtml). Molybdenum with its unique electrical and thermal conductivity is widely used in national defense, super alloy, iron and steel metallurgy, electrical and chemical industry, aerospace and other fields. The United States, Canada, Japan, South Korea and other countries have established molybdenum national strategic reserve (see report from Hebei Province Department of Natural Resources, of which the link is: https://zrzy.hebei.gov.cn/heb/gongk/gkml/kjxx/kpyd/10971182134109683712.html).

The research team of the University of Exeter in the UK found that people with high tungsten content in the body would greatly increase the probability of stroke (see Metal Encyclopedia for details, of which the link is: http://baike.asianmetal.cn/metal/w/health.shtml), and people with high molybdenum content would have diseases such as joint swelling, deformation, kidney damage, growth retardation, hair loss, arteriosclerosis, and connective tissue disease (see Metal Encyclopedia for details, of which the link is: http://baike.asianmetal.cn/metal/mo/health.shtml). The World Health Organization's “Guidelines for Drinking Water Quality” and China's “Environmental Quality Standards for Surface Water” stipulate that the molybdenum mass effluent limit is 0.07 mg/L, which is an extremely high regulation.

In the patent method embodiment of WO2021002484, the tungsten-based catalyst used in large amounts, up to 0.03-0.05 equivalents (the molar amount of tungsten element acting as catalyst versus the molar amount of the raw material Compound I, the same meaning below). The molybdenum-based catalyst is used in larger amounts, up to 0.07 equivalents (calculated based on the molar amount of molybdenum element acting as catalyst versus the molar amount of the raw material Compound I, the same meaning below). According to the data reported in the patent, to synthesize one ton of Compound II, the consumption of sodium tungstate dihydrate is about 26.4 kg (calculated according to the data in its Example 2-14, which is the minimum amount of catalyst used in all examples of WO2021002484); the consumption of ammonium molybdate tetrahydrate is about 32.9 kg (calculated according to the data in its Example 2-17). On one hand, these demonstrate the method of WO2021002484 lead to the large consumption of scarce strategic metal resources of either tungsten or molybdenum; on the other hand, there must be large amount of toxic inorganic waste salts of tungsten or molybdenum in the wastewater at the end of the reaction, which has higher potential hazard to the human body and the environment. That kind of wastewater must be treated at a higher cost for harmless treatment, otherwise industrial production is not sustainable. At the same time, the solvent used in the preparation of Compound II in the patent method is alcohol, nitrile, amide, ester, sulfone, most of these solvents are miscible with water and difficult to recover and reuse after the reaction is completed (for example, methanol, ethanol, acetonitrile, dimethyl formamide, N-methyl pyrrolidone, sulfolane, acetic acid used in its examples), or because of their larger water solubility, resulting in more solvent loss due to dissolution in the aqueous phase at the end of the reaction (for example, butanol, 2-propanol, tert-butyl alcohol, ethyl acetate used in its examples), these two factors will also lead to the difficulty and high cost of wastewater treatment due to the presence of a large amount of organic solvents in the wastewater.

An improved preparation method of Compound II is disclosed in CN115776978A, which is improved on the basis of the preparation method reported in WO2021002484, mainly in the use of carboxylic acid assisted catalytic oxidation; the catalysts are mainly sodium tungstate and ammonium molybdate, the amount of tungsten catalyst used is 0.02-0.03 equivalents, and the amount of molybdenum catalyst used is as high as 0.07 equivalents; the solvent used in the examples is acetic acid, acetonitrile or methanol; the highest yield obtained by HIPLC external standard test is 98.1%. It can be seen that, compared with the method in WO2021002484, the preparation method of Compound II reported in CN115776978A has slightly improved yield (increased by 2.2%), but still has the problems or defects in the preparation method of WO2021002484 as described above.

Similarly, the preparation methods of Compound II are disclosed in patents CN117794925A, CN117440754A, CN111574511A and CN114929693A, but they all have the problems or defects in the preparation method of WO2021002484 completely or mostly.

Therefore, there is an urgent need in the art to develop a method for preparing Compound II more suitable for industrialization, which consumes less scarce strategic metal resources, is safe and environmentally friendly, and the solvent is easy to be recycled and reused, so that more efficient catalysts can be used, thereby reducing the consumption of scarce metal resources, reducing the potential harm to human body and environment, making the solvent easy to be recycled and reused, generating less waste, being easier to treat, and having lower treatment cost.

### SUMMARY

The present application is to provide a method for preparing Compound II, which greatly reduces the amount of catalyst used.

The present application provides a method for preparing Compound II, and the method includes: using Compound I as a raw material, performing a reaction under the action of an organometallic catalyst and a phase transfer catalyst, and oxidizing with hydrogen peroxide to obtain Compound II. The reaction formula is as follows:

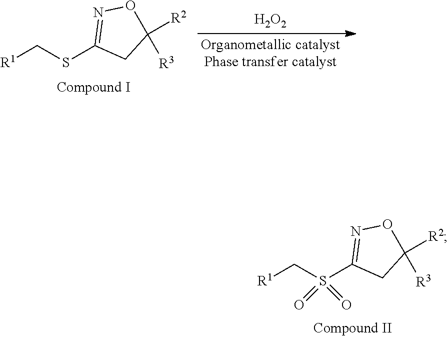

wherein R¹ is 1-methyl-3-trifluoromethyl-5-difluoromethoxy-1H-pyrazol-4-yl, and R² and R³ are each methyl.

The organometallic catalyst and Compound I have a molar ratio of (0.0001-0.018):1, which may be, for example, 0.0001:1, 0.0005:1, 0.0008:1, 0.001:1, 0.002:1, 0.003:1, 0.004:1, 0.005:1, 0.008:1, 0.010:1, 0.012:1, 0.015:1, 0.018:1, etc.

Preferably, the organometallic catalyst is an organotungsten catalyst and an organomolybdenum catalyst.

Preferably, the organotungsten catalyst includes any one or a combination of at least two of hexacarbonyltungsten, tungsten (V) ethoxide, tungsten (VI) ethoxide, tungsten (VI) isopropoxide, bis(tert-butylimido)bis(dimethylamine)tungsten (VI), trimethylphenyltungsten tricarbonyl, bis(acetonitrile) tetracarbonyltungsten, (1,1-bis(diphenylphosphino)ferrocene) tetracarbonyltungsten, and the like organic tungsten-containing metal compounds, and further preferably hexacarbonyltungsten. The hexacarbonyltungsten used in the present application can be directly purchased and used; there are 91 known domestic suppliers and 44 known foreign suppliers, as shown in Chemical Book (https://www.chemicalbook.com/ProductList.aspx?cbn=CB6684220).

Preferably, the organomolybdenum catalyst includes any one or a combination of at least two of hexacarbonylmolybdenum, molybdenum acetate and its polymers, molybdenum iso-octoate, molybdenum di(ethylbenzene), molybdenum(V) isopropoxide, bis(t-butylimido)bis(dimethylamino)molybdenum(VI), tris(acetonitrile)molybdenum tricarbonyl, molybdenum(V) trichloroisopropanolate, (propylcyclopentadienyl)molybdenum(I) tricarbonyl dimer, (1,1′-bis(diphenylphosphino)ferrocene) molybdenum tetracarbonyl, and the like organic molybdenum-containing metal compounds, and further preferably hexacarbonylmolybdenum.

Preferably, the organometallic catalyst and Compound I has a molar ratio of (0.003-0.018):1, more preferably (0.005-0.015):1 (corresponding to hexacarbonyltungsten consumption of 4.6-13.9 kg of hexacarbonyltungsten to prepare one ton of Compound II), more preferably 0.003:1, more preferably 0.004:1, more preferably 0.005:1, more preferably 0.008:1, more preferably 0.010:1, more preferably 0.012:1, and further more preferably 0.015:1, i.e. about 4.6 kg of hexacarbonyltungsten is consumed to prepare one ton of Compound II (calculated according to the data in Example 58 of this application, which is 82.6% less than the mass of the inorganic tungsten salt catalyst described in Example 2-14 of WO2021002484).

Preferably, the phase transfer catalyst (PTC for short) is selected from any one or a combination of at least two of polyethers, crown ethers, quaternary ammonium salts, quaternary ammonium bases or quaternary phosphonium salts, and further preferably quaternary ammonium salts.

Preferably, the quaternary ammonium salt includes any one or a combination of at least two of benzyltriethylammonium chloride (TEBA for short), tetrabutylammonium bromide (TBAB for short), tetrabutylammonium chloride, tetrabutylammonium hydrogen sulfate, trioctylmethylammonium chloride, dodecyltrimethylammonium chloride, tetradecyltrimethylammonium chloride or tetramethylammonium chloride (TMAC for short), and further preferably tetrabutylammonium bromide.

Preferably, the phase transfer catalyst and Compound I has a molar ratio of (0.005-0.02):1, which may be, for example, 0.005:1, 0.010:1, 0.012:1, 0.015:1, 0.018:1, 0.02:1, etc., preferably (0.005-0.018):1, and more preferably 0.01:1.

Preferably, the reaction is carried out in the presence of an acid.

Preferably, the acid is an organic acid, an inorganic acid or a substance capable of reacting with water in situ to form an acid; the reaction of the present application can be carried out in the presence of an organic acid or an inorganic acid or a substance capable of reacting with water in situ to form an acid. The presence of an acid has a certain positive effect on improving the performance of the catalyst, but it is not necessary. The reaction of the present application can be carried out efficiently in the absence of an acid.

Preferably, the organic acid is selected from any one or a combination of at least two of formic acid, acetic acid, difluoroacetic acid, trifluoroacetic acid, dichloroacetic acid or trichloroacetic acid.

Preferably, the inorganic acid is selected from any one or a combination of at least two of sulfuric acid, hydrochloric acid, nitric acid, phosphoric acid, boric acid, chlorosulfonic acid or chlorous sulfonic acid.

Preferably, the substance capable of reacting with water in situ to form an acid is selected from any one or a combination of at least two of sulfuryl chloride, thionyl chloride, acetyl chloride, dichloroacetyl chloride, trichloroacetyl chloride, trifluoroacetyl chloride, trifluoroacetyl fluoride, acetic anhydride, trifluoroacetic anhydride, peroxyacetic acid, triphosgene, chlorine, or bromine.

Preferably, a molar ratio of the acid or the substance capable of reacting in situ with water to form an acid to Compound I is (0.001-1):1, which may be, for example, 0.001:1, 0.010:1, 0.030:1, 0.100:1, 0.300:1, 0.500:1, 0.800:1, 1.000:1, etc., preferably (0.01-0.3):1, and more preferably 0.1:1.

Preferably, the hydrogen peroxide is aqueous hydrogen peroxide solution and has a concentration of 10-70%, preferably 20-50%, more preferably 30-50%, and further more preferably 50%.

Preferably, the hydrogen peroxide and Compound I has a molar ratio of (2.0-6.0):1, preferably (2.05-4.0):1, more preferably (2.05-3.00):1, more preferably (2.0-2.1):1, and further more preferably 2.05:1.

Preferably, the hydrogen peroxide is added to the reaction system in a dropwise manner.

Preferably, the dropwise addition is conducted over a period of 0.5-3 h, which may be, for example, 0.5 h, 1 h, 1.5 h, 2 h, 2.5 h or 3 h, and other specific addition time between 0.5 h and 3 h; limited by the length of the present application and for the sake of brevity, the other addition time included in the range will not be listed any more.

In the present application, the reaction is carried out in an organic solvent, and the organic solvent is immiscible with water.

Preferably, the organic solvent is selected from any one or a combination of at least two of halogenated aliphatic hydrocarbons, aromatic hydrocarbons or halogenated aromatic hydrocarbons.

Preferably, the organic solvent includes but is not limited to any one or a combination of at least two of 1,2-dichloroethane, dichloromethane, chloroform, 1,1,2-trichloroethane, toluene, xylene, mesitylene, chlorobenzene or dichlorobenzene, etc., further preferably 1,2-dichloroethane.

Preferably, the reaction is carried out at a temperature of 30-90° C., which may be, for example, 30° C., 40° C., 50° C., 60° C., 70° C., 80° C. or 90° C., and other specific degrees between 30° C. and 90° C.; limited by the length of the present application and for the sake of brevity, will not list the other specific degrees included in the range will not be listed any more, preferably 40-60° C., and more preferably 50-60° C.

Preferably, the reaction is carried out for a period of 2.5-12 h, which may be, for example, 2.5 h, 4 h, 6 h, 8 h, 10 h or 12 h, and other specific time between 2.5 h and 12 h; limited by the length of the present application and for the sake of brevity, the other specific time included in the range will not be listed any more.

Preferably, the reaction is also followed by a work-up step comprising: adding water, recovering the solvent by atmospheric distillation, adding methanol, stirring the mixture to crystallize the crude Compound II, filtrating the mixture, washing the filter cake with water, and drying the wet filter cake to obtain high-quality Compound II (sulfoxide <0.2%, “sulfoxide” is the abbreviation, and its molecular structure is described as the “sulfoxide” impurity in the reaction formula in the “DETAILED DESCRIPTION” section below). As reported, it is not easy to prepare high-quality Compound II with a sulfoxide content of <0.2%, which is desired by the industry and monitored by governments. For example: In CN112969697A, on page 132, paragraph [1759], it is stated and summarized that “In the production method described in Japanese Kokai 2013-512201 (JP2013-512201 A) (Patent literature Reference 7), even after adding an excess of hydrogen peroxide and aging for 16 hours, the compound (6a, which is abbreviated as sulfoxide in this application) remained as a reaction intermediate at a ratio of 5.0%. In addition, even after purification, the ratio of the compound (6a) did not decrease. It was again confirmed that it is difficult to purify the compound of formula (5) by separating the compound of formula (5) and the compound of formula (6).” In CN117794925A, in paragraph [0012], it is stated that “the compound of formula (III) (i.e., abbreviated as sulfoxide in this application) remains as a byproduct in the final product, as Compound II is very difficult to remove due to structural similarity. However, if the compound of formula (III) is not separated, it can lead to deterioration in the quality of the Compound II preparation and can also cause phytotoxicity to crops. Moreover, if such impurity is not removed/controlled, regulatory issues can arise.”

As a preferred technical solution of the present application, the method for preparing Compound II includes the following steps:
adding hydrogen peroxide dropwise to an organic solvent system containing Compound I, an organometallic catalyst and a phase transfer catalyst, reacting at 30-90° C. for 2.5-12 hours, adding water and performing atmospheric pressure distillation to recover the solvent, adding methanol and stirring for crystallization, filtering, water washing, and drying to obtain Compound II;
wherein the organometallic catalyst is an organotungsten catalyst and organomolybdenum catalyst, and the organometallic catalyst and Compound I has a molar ratio of (0.0001-0.018):1;
the phase transfer catalyst is selected from any one or a combination of at least two of polyethers, crown ethers, tertiary amines, quaternary ammonium salts, quaternary ammonium hydroxides, or quaternary phosphonium salts, and the phase transfer catalyst and Compound I has a molar ratio of (0.005-0.02):1;
the hydrogen peroxide is aqueous hydrogen peroxide solution and has a concentration of 10-70%, and hydrogen peroxide and Compound I has a molar ratio of (2.0-6.0):1;
the organic solvent is selected from any one or a combination of at least two of halogenated aliphatic hydrocarbons, aromatic hydrocarbons, or halogenated aromatic hydrocarbons.

Compared with the prior art, the present application has the following beneficial effects:

The method for preparing Compound II provided by the present application uses a catalyst that has extremely high activity for the oxidation of sulfide compounds to prepare sulfone compounds, greatly reduces the amount of catalyst used, saves non-renewable and scarce strategic resources, and greatly reduces the risk of harm to the natural environment and human health.

The present application uses an organic solvent that is immiscible with water as the solvent, making it easy to separate and recover, and reducing treatment costs.

The overall process provided by the present application is more efficient, consumes fewer resources, generates less waste, and has lower waste treatment costs, and can produce Compound II in high yield (>98%), high purity (>99%) and high quality (sulfoxide <0.2%).

The method for preparing Compound II provided by the present application is more environmentally friendly, economical, safe and controllable, so it has high industrialization feasibility and broad industrial application prospects and social value.

### BRIEF DESCRIPTION OF DRAWINGS

FIG. 1 shows a Compound II Intermediate Control Analysis HPLC chromatogram of Example 1.

FIG. 2 shows a Compound II Intermediate Control Analysis HPLC chromatogram of Example 12.

FIG. 3 shows a Compound II Intermediate Control Analysis HPLC chromatogram of Example 19.

FIG. 4 shows a Compound II Intermediate Control Analysis HPLC chromatogram of Example 20.

FIG. 5 shows a Compound II Intermediate Control Analysis HPLC chromatogram of Comparative Example 1.

FIG. 6 shows a reaction system state at the time when hydrogen peroxide was added dropwise in the experiment of Comparative Example 1.

FIG. 7 shows a reaction system state after heating the reaction for 5 hours in the experiment of Comparative Example 1.

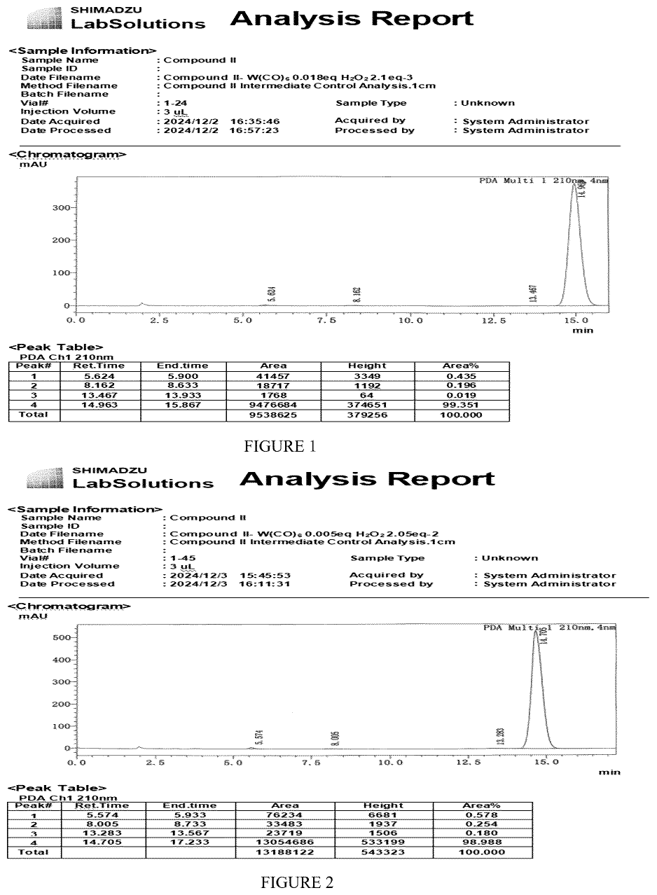

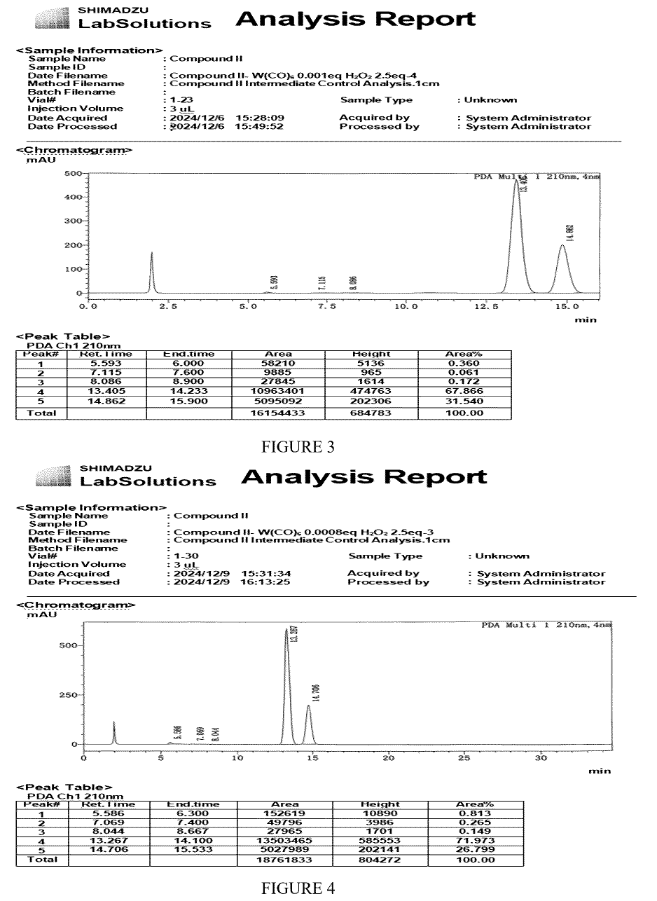

### DETAILED DESCRIPTION

The technical solutions of the present application are further illustrated below through specific embodiments. Those skilled in the art should understand that the embodiments are only for the purpose of understanding the present application, and should not be regarded as specific limitations to the present application.

As shown in the following examples, the method for preparing Compound II uses Compound I as the raw material, in the water-immiscible organic solvent, under the action of the organometallic catalyst and the phase transfer catalyst, and under the oxidation of hydrogen peroxide to afford Compound II in high yield, high purity and high quality. The reaction formula is as follows:

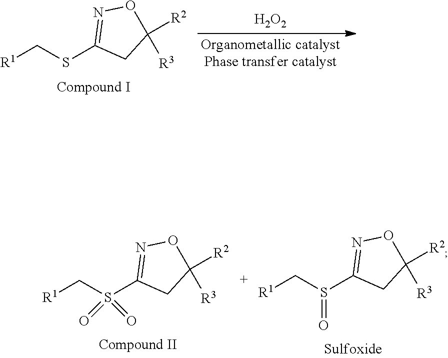

in the formula, R¹ is 1-methyl-3-trifluoromethyl-5-difluoromethoxy-1H-pyrazol-4-yl, and R² and R³ are each methyl.

In the following specific embodiments of the present application, all raw materials without preparation methods were purchased directly from market, the purity of raw materials and products refers to mass percentage, the reaction conversion rate was determined by HPLC area normalization method, the purity (content) of the product was determined by HPLC external standard method, and the yield was mass yield. The molecular structure of the product was characterized and confirmed by UHPLC-MS and/or ¹H-NMR.

### Example 1

Into a reaction kettle, 100 g of dichloroethane was added and stirring was started. Then 6 g of aqueous sulfuric acid solution (purity 49%, 0.03 mol), 0.33 g of tetra-n-butylammonium bromide (purity 99%, 0.001 mol), 0.64 g of hexacarbonyltungsten (purity 99%, 0.0018 mol) and 36.4 g of Compound I (purity 98.7%, 0.1 mol) were added successively. The temperature was raised to 60° C. and 23.8 g of aqueous hydrogen peroxide (concentration 30%, 0.21 mol) was added dropwise within 1 h. After the dropwise addition was completed, the reaction was maintained at the temperature for 3 h. After 3 h, the Compound II Intermediate Control Analysis HPLC chromatogram showed that the residual Compound I was 0% (HPLC area normalization, 210 nm), the residual sulfoxide was 0.019% (HPLC area normalization, 210 nm, retention time 13.467 min, see FIG. 1 for details), and Compound II was 99.35% (HPLC area normalization, 210 nm, retention time 14.963 min, see FIG. 1 for details). 30 g of water was added to the system. 110 g of mixed solvents of dichloroethane and water were removed at a vacuum of 20.5 kPa and an internal temperature of about 60° C., followed by addition of 50 g of methanol and agitation for 30 min. The methanolic mixture was cooled to 0° C. and stirred for 30 min. A large amount of solid precipitated, which was filtered. The filter cake was washed with 30 g of water with stirring, filtered and dried (5 kPa, 60° C.) to obtain 38.9 g of Compound II (white crystal), with a purity of 99.20 and a yield of 98.6%. No sulfoxide was detected in the product. An appropriate amount of the obtained Compound II was taken for analysis and the following data were obtained: UHIPLC-MS (m/z, ESI): 392.0693 (M+H)⁺, theoretical value: 392.0698; 1H-NMR (DMSO, 400 MHz) δ (ppm): 1.520 (s, 6H, 2CH₃), 3.106 (s, 2H, CH₂), 3.878 (s, 3H, CH₃), 4.602 (s, 2H, CH₂), 6.831 (t, J=72 Hz, 1H, CF₂H).

### Examples 2-22

Examples 2-22, as shown in Table 1, are the same as Example 1 except that they used the respective catalyst equivalent, aqueous hydrogen peroxide concentration, hydrogen peroxide equivalent and reaction time, which are listed in Table 1 together with the corresponding results. For ease of analysis, the results of Example 1 are also listed in Table 1.

TABLE 1

| | | | | Reaction | Compound | Sulfoxide | Compound | Compound | Sulfoxide in |
|---|---|---|---|---|---|---|---|---|---|
| | Hexacarbonyltungsten | H₂O₂ | H₂O₂ | time | I area^{a} | area^{b} | II purity | II yield | Compound II |
| Example | (mol %) | (%) | (mol %) | (h) | (%) | (%) | (%) | (%) | (%) |
| 1 | 1.8 | 30 | 210 | 3 | 0 | 0.019 | 99.2 | 98.6 | 0 |
| 2 | 1.8 | 50 | 200 | 8 | 0 | 2.4 | 97.0 | 96.2 | 2.2 |
| 3 | 1.8 | 50 | 205 | 3 | 0 | 0.12 | 99.1 | 98.5 | 0 |
| 4 | 1.5 | 50 | 205 | 3 | 0 | 0.13 | 99.2 | 98.4 | 0 |
| 5 | 1.2 | 50 | 205 | 3.5 | 0 | 0.13 | 99.1 | 98.5 | 0 |
| 6 | 1 | 20 | 210 | 6 | 0 | 0.17 | 99.0 | 98.4 | 0.01 |
| 7 | 1 | 30 | 210 | 4 | 0 | 0.11 | 99.1 | 98.5 | 0 |
| 8 | 1 | 40 | 210 | 3.5 | 0 | 0.09 | 99.2 | 98.4 | 0 |
| 9 | 1 | 50 | 210 | 2.5 | 0 | 0.04 | 99.1 | 98.5 | 0 |
| 10 | 0.5 | 30 | 210 | 6 | 0 | 0.18 | 99.3 | 98.5 | 0.01 |
| 11 | 0.5 | 50 | 210 | 4.5 | 0 | 0.13 | 99.2 | 98.4 | 0.01 |
| 12 | 0.5 | 50 | 205 | 5 | 0 | 0.18 | 99.3 | 98.5 | 0.02 |
| 13 | 0.5 | 50 | 200 | 8 | 0 | 3.7 | 95.6 | 94.8 | 3.5 |
| 14 | 0.4 | 30 | 230 | 8 | 0 | 0.15 | 99.1 | 98.5 | 0 |
| 15 | 0.4 | 50 | 220 | 8 | 0 | 0.17 | 99.3 | 98.5 | 0 |
| 16 | 0.3 | 50 | 300 | 12 | 0 | 0.27 | 99.0 | 98.4 | 0.08 |
| 17 | 0.2 | 30 | 400 | 12 | 0 | 58.4 | 41.1 | 40.3 | 58.2 |
| 18 | 0.2 | 50 | 250 | 12 | 0 | 51.1 | 49.2 | 48.7 | 49.9 |
| 19 | 0.1 | 50 | 250 | 12 | 0 | 67.87 | 31.4 | 30.8 | 67.7 |
| 20 | 0.08 | 50 | 250 | 12 | 0 | 71.97 | / | / | / |
| 21 | 0.05 | 50 | 250 | 12 | 2.7 | 90.3 | / | / | / |
| 22 | 0.01 | 50 | 250 | 12 | 37.6 | 60.7 | / | / | / |

Comments:
^{a}Compound I area refers to Compound I area normalization in Compound II Intermediate Control Analysis HPLC chromatogram;
^{b}sulfoxide area refers to sulfoxide area normalization in Compound II Intermediate Control Analysis HPLC chromatogram.
The “^{a}” and “^{b}” below share the same comments herein.

### Examples 23-36

Examples 23-36, as shown in Table 2, are the same as Example 11 except that they used the respective acid and its equivalent, the respective phase transfer catalyst and its equivalent, and the respective reaction time, which are listed in Table 2 together with the corresponding results. For ease of analysis, the results of Example 11 are also shown in Table 2.

TABLE 2

| | | Quantity | | PTC | Reaction | Compound | Sulfoxide | Compound | Compound | Sulfoxide in |
|---|---|---|---|---|---|---|---|---|---|---|
| | | of acid | | used | time | I area^{a} | area^{b} | II purity | II yield | Compound II |
| Example | Acid | (mol %) | PTC | (mol %) | (h) | (%) | (%) | (%) | (%) | (%) |
| 11 | sulfuric | 30 | TBAB | 1 | 4.5 | 0 | 0.13 | 99.2 | 98.4 | 0.01 |
| | acid | | | | | | | | | |
| 23 | sulfuric | 30 | TMAC | 1 | 5 | 0 | 0.13 | 99.2 | 98.4 | 0 |
| | acid | | | | | | | | | |
| 24 | sulfuric | 30 | TBAB | 2 | 4 | 0 | 0.17 | 99.2 | 98.4 | 0.01 |
| | acid | | | | | | | | | |
| 25 | sulfuric | 30 | TBAB | 1.8 | 4 | 0 | 0.11 | 99.2 | 98.4 | 0 |
| | acid | | | | | | | | | |
| 26 | sulfuric | 30 | TBAB | 1.5 | 4.5 | 0 | 0.13 | 99.0 | 98.4 | 0 |
| | acid | | | | | | | | | |
| 27 | sulfuric | 30 | TBAB | 0.5 | 6 | 0 | 0.21 | 99.1 | 98.5 | 0.03 |
| | acid | | | | | | | | | |
| 28 | sulfuric | 30 | TBAB | 0.3 | 8 | 0 | 7.20 | 92.1 | 91.3 | 6.97 |
| | acid | | | | | | | | | |
| 29 | sulfuric | 30 | TBAB | 0.1 | 8 | 0 | 23.4 | 76.0 | 75.2 | 23.1 |
| | acid | | | | | | | | | |
| 30 | sulfuric | 30 | TBAB | 0 | 6 | 67.3 | 30.4 | / | / | / |
| | acid | | | | | | | | | |
| 31 | sulfuric | 100 | TBAB | 1 | 4 | 0 | 0.11 | 99.2 | 98.4 | 0 |
| | acid | | | | | | | | | |
| 32 | sulfuric | 10 | TBAB | 1 | 5 | 0 | 0.09 | 99.3 | 98.5 | 0 |
| | acid | | | | | | | | | |
| 33 | sulfuric | 3 | TBAB | 1 | 6 | 0 | 0.11 | 99.0 | 98.7 | 0 |
| | acid | | | | | | | | | |
| 34 | sulfuric | 0.1 | TBAB | 1 | 8 | 0 | 0.21 | 99.2 | 98.4 | 0.02 |
| | acid | | | | | | | | | |
| 35 | sulfuric | 0 | TBAB | 1 | 8 | 0 | 0.23 | 99.1 | 98.5 | 0.04 |
| | acid | | | | | | | | | |
| 36 | acetic | 30 | TBAB | 1 | 6 | 0 | 0.13 | 99.1 | 98.3 | 0 |
| | acid | | | | | | | | | |

Examples 37-42, as shown in Table 3, are the same as Example 11 except that they used the respective solvent and its quantity, the respective reaction temperature, and the respective reaction time, which are listed in Table 3 together with the corresponding results. For ease of analysis, the results of Example 11 are also listed in Table 3.

TABLE 3

| | | Solvent | Reaction | Reaction | Compound | Sulfoxide | Compound | Compound | Sulfoxide in |
|---|---|---|---|---|---|---|---|---|---|
| | | quantity | temperature/ | time | I area^{a} | area^{b} | II purity | II yield | Compound II |
| Example | Solvent | (kg/mol) | ° C. | (h) | (%) | (%) | (%) | (%) | (%) |
| 11 | dichloroethane | 1.0 | 60 | 4.5 | 0 | 0.13 | 99.2 | 98.4 | 0.01 |
| 37 | toluene | 1.0 | 60 | 6 | 0 | 0.15 | 99.1 | 98.5 | 0.01 |
| 38 | dichloromethane | 2.0 | 40 | 10 | 0 | 0.13 | 99.0 | 98.4 | 0 |
| 39 | trichloromethane | 1.0 | 50 | 6 | 0 | 0.14 | 99.2 | 98.6 | 0 |
| 40 | dichloroethane | 1.0 | 55 | 4.5 | 0 | 0.11 | 99.2 | 98.1 | 0 |
| 41 | dichloroethane | 1.0 | 70 | 8 | 0 | 3.2 | 96.3 | 95.7 | 2.9 |
| 42 | dichloroethane | 1.0 | 80 | 8 | 0 | 13.7 | 85.6 | 84.7 | 13.5 |

Note:
kg/mol in the table represents the quantity of solvent versus per molar Compound I.

### Examples 43-48

Examples 43-48, as shown in Table 4, are the same as Example 35 except that they used the respective amount of hexcarbonyltungsten, the respective amount of 50% of aqueous hydrogen peroxide, the respective drop-adding time of aqueous hydrogen peroxide and the reaction time, which are listed in Table 4 together with the corresponding results. For ease of analysis, the results of Example 35 are also listed in Table 4.

TABLE 4

| | | | Drop- | | Compound | Sulfoxide | Compound | Compound | Sulfoxide in |
|---|---|---|---|---|---|---|---|---|---|
| | Hexacarbonyltungsten | H₂O₂ | adding | Reaction | I area^{a} | area^{b} | II purity | II yield | Compound II |
| Example | (mol %) | (mol %) | time (h) | time (h) | (%) | (%) | (%) | (%) | (%) |
| 35 | 0.5 | 210 | 1 | 8 | 0 | 0.23 | 99.1 | 98.5 | 0.04 |
| 43 | 1.8 | 210 | 0.5 | 5 | 0 | 0.09 | 99.2 | 98.4 | 0 |
| 44 | 1.5 | 210 | 1 | 5 | 0 | 0.11 | 99.3 | 98.5 | 0 |
| 45 | 1.2 | 210 | 1 | 6 | 0 | 0.11 | 99.2 | 98.4 | 0 |
| 46 | 1 | 210 | 1 | 6.5 | 0 | 0.13 | 99.1 | 98.5 | 0 |
| 47 | 0.4 | 250 | 2 | 10 | 0 | 0.19 | 99.0 | 98.4 | 0.02 |
| 48 | 0.3 | 400 | 3 | 10 | 0 | 0.21 | 99.1 | 98.5 | 0.03 |

As can be seen from the data in Table 4, without the use of acid, high quality Compound II can be obtained while 0.3%-1.8 mol % equivalents of hexacarbonyltungsten are employed.

### Examples 49-57

Examples 49-57, as shown in Table 5, are the same as Example 9 except that they used the respective organometallic catalyst and its quantity, and the respective reaction time, which are listed in Table 5 together with the corresponding results. For ease of analysis, the results of Example 9 and Example 11 are also listed in Table 5.

TABLE 5

| | | CAT | Reaction | Compound | Sulfoxide | Compound | Compound | Sulfoxide in |
|---|---|---|---|---|---|---|---|---|
| | | quantity | time | I area^{a} | area^{b} | II purity | II yield | Compound II |
| Example | CAT | (mol %) | (h) | (%) | (%) | (%) | (%) | (%) |
| 9 | Hexacarbonyltungsten | 1 | 2.5 | 0 | 0.04 | 99.1 | 98.5 | 0 |
| 11 | Hexacarbonyltungsten | 0.5 | 4.5 | 0 | 0.13 | 99.2 | 98.4 | 0.01 |
| 49 | Tungsten(VI) | 1 | 2.5 | 0 | 0.17 | 99.3 | 98.5 | 0 |
| | ethoxide | | | | | | | |
| 50 | Tungsten(VI) | 0.5 | 4.5 | 0 | 0.23 | 99.1 | 98.3 | 0.04 |
| | ethoxide | | | | | | | |
| 51 | cat1 | 1 | 3 | 0 | 0.27 | 99.1 | 98.3 | 0.08 |
| 52 | cat1 | 0.5 | 6 | 0 | 2.6 | 97.0 | 96.2 | 2.3 |
| 53 | cat2 | 1 | 3 | 0 | 3.7 | 96.1 | 95.3 | 3.4 |
| 54 | cat2 | 0.5 | 6 | 0 | 7.5 | 91.7 | 90.9 | 7.3 |
| 55 | Hexacarbonylmolybdenum | 1 | 6 | 0 | 24.3 | / | / | / |
| 56 | Cat3 | 0.5 | 6 | 0 | 26.5 | / | / | / |
| 57 | Cat4 | 1 | 6 | 0 | 27.2 | / | / | / |

Note:
In Table 5, CAT refers to organometallic catalyst, cat1 refers to tricarbonyl trimethylphenyl tungsten, cat2 refers to (1,1-bis(diphenylphosphono)ferrocene)tetracarbonyl tungsten, cat3 refers to molybdenum(II) acetate dimer, and cat4 refers to (1,1′-bis(diphenylphosphino)ferrocene)tetracarbonyl molybdenum.

### Example 58

100 g of dichloroethane was added to the reaction kettle and stirring was initiated. 2 g of sulfuric acid aqueous solution (49% purity, 0.01 mol), 0.33 g of tetrabutylammonium bromide (99% purity, 0.001 mol), 0.18 g of hexacarbonyltungsten (99% purity, 0.0005 mol), and 36.4 g of Compound I (98.7% purity, 0.1 mol) were sequentially added. The mixture was heated to 55° C. and 13.9 g of aqueous hydrogen peroxide (concentration 50%, 0.205 mol) was added dropwise within 1 hour. After completion of the addition, the reaction was maintained at 55° C. for 5.5 hours. At 5.5 hours, the Compound II Intermediate Control Analysis IPLC chromatogram showed that the residual sulfoxide was 0.18%. The reaction was stopped. 30 g of water was added to the system. 110 g of mixed solvents of dichloroethane and water were removed under vacuum (20.5 kPa) at an internal temperature of about 60° C. (98.7 g of dichloroethane recovered via phase separation of the mixed solvents), followed by adding 50 g of methanol. The mixture was stirred for 30 minutes and cooled to 0° C. and stirred for an additional 30 minutes, resulting in plenty of precipitate. The precipitate was filtered washed with 30 g of water under stirring and dried (5 kPa, 60° C.) to afford 38.9 g of Compound II (white crystals) with a purity of 99.2%, a yield of 98.6%, and a sulfoxide content of 0.02%.

### Example 59

Example 59 is the same as Example 58 except that the total dichloroethane (100 g) of Example 59 was composed of 98.7 g of dichloroethane recovered from Example 58 and 1.3 g of fresh dichloroethane. After 5.5 h of reaction, the Compound II Intermediate Control Analysis HPLC chromatogram showed that the remaining Compound I was 0% (HPLC area normalization, 210 nm) and the remaining sulfoxide was 0.12% (HPLC area normalization, 210 nm). After workup 38.8 g of Compound II was obtained with 99% purity and 98.4% yield. No sulfoxide was detected in the product. This demonstrated that the recovered dichloroethane can be used directly in the preparation of new batch of Compound II.

### Comparative Example 1

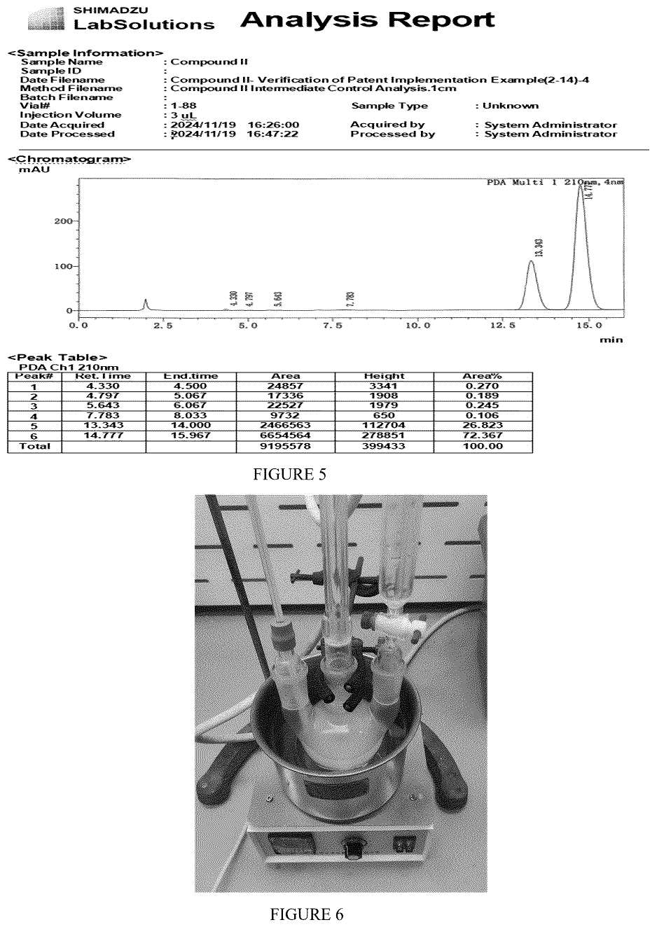

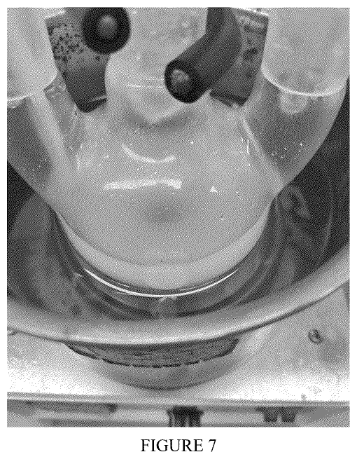

Among methods for preparing Compound II disclosed in WO2021002484, the highest yield of Compound II (i.e., 95.9%) was obtained according to the method of Example 2-14, in which the catalyst was sodium tungstate dihydrate, the catalyst was 0.03 mol equivalent of Compound I, hydrogen peroxide was 2.3 equivalents of Compound I, and the reaction temperature was 75-80° C. After holding the reaction for 5 h, the residual sulfoxide was 0.5% and the final product yield was 95.9% after workup. The present applicant repeated Example 2-14 of WO2021002484 in accordance with its description (FIG. 6 and FIG. 7 are reaction states at two important stages).

Under the protection of nitrogen, 88.9 g of acetonitrile solution of Compound I (40.4% purity, 0.1 mol, the other is acetonitrile), 10 g of water and 1.0 g of sodium tungstate dihydrate (99% purity, 0.003 mol) were added to the reaction kettle. The stirring was turned on and the temperature of the internal kettle was raised to 75-80° C. 22.4 g of aqueous hydrogen peroxide (concentration 35%, 0.23 mol) was dropped into the reaction kettle during 1 h. After drop-adding aqueous hydrogen peroxide completed, the reaction was held at 75-80° C. for 5 h. After 5 h of reaction, Compound II Intermediate Control Analysis HPLC chromatogram showed that the remaining Compound I was 0% (HPLC area normalization, 210 nm), the remaining sulfoxide was 26.82% (HPLC area normalization, 210 nm, retention time 13.343 min, see FIG. 5 for details), and Compound II was 72.37% (HPLC area normalization, to 210 nm, retention time 14.777 min, see FIG. 5 for details). 22.2 g of aqueous sodium sulfite (17%, 0.03 mol) was added to the reaction and the resultant mixture was stirred at an internal temperature of 55-65° C. for 30 min. Then the stirring was stopped, the aqueous phase was removed, the oily phase was concentrated after adding 50 ml of water under reduced pressure, and the residue of the kettle was crystallized by adding 80 ml of isopropanol at room temperature. After filtration, washing with 10 ml of isopropanol and 10 ml of water and drying (5 kPa, 60° C.), 38.8 g of Compound II (white crystal) was obtained in purity of 72.5%, yield of 71.9% and the sulfoxide content of 26.6%.

### Comparative Example 2

On the basis of Comparative Example 1, the reaction temperature was lowered to 58° C. and appropriate amount of sulfuric acid was added as follows:

Under the protection of nitrogen, 88.9 g of acetonitrile solution of Compound I (purity 40.4%, 0.1 mol, the other is acetonitrile), 10 g of water, 6 g of aqueous sulfuric acid (49%, 0.03 mol) and 1.0 g of sodium tungstate dihydrate (purity 99%, 0.003 mol) were added to the reaction kettle. The stirring was turned on and the temperature of the kettle was raised to 55-60° C. 22.4 g of aqueous hydrogen peroxide (concentration 35%, 0.23 mol) was dropped into the reaction kettle during 1 h. After drop-adding aqueous hydrogen peroxide completed, the reaction was held at 55-60° C. for 5 h. After 5 h of reaction, Compound II Intermediate Control Analysis IPLC chromatogram showed that the remaining Compound I was 0% and the remaining sulfoxide was 0.33% (both are IPLC area normalizations, 210 nm). 22.2 g of aqueous sodium sulfite (17%, 0.03 mol) was added to the reaction and the resultant mixture was stirred at an internal temperature of 55-65° C. for 30 min. Then the stirring was stopped, the aqueous phase was removed, the oily phase was concentrated after adding 50 ml of water under reduced pressure, and the residue of the kettle was crystallized by adding 80 ml of isopropanol at room temperature. After filtration, washing with 10 ml of isopropanol and 10 ml of water and drying (5 kPa, 60° C.), 38.1 g of Compound II (white crystal) was obtained in purity of 99.0%, yield of 96.4% and the sulfoxide content of 0.12%. The experimental results were significantly improved, but the catalyst dosage was obviously increased comparing with the present application (the molar equivalent was 166.7% of the maximum catalyst dosage of the present application).

### Comparative Examples 3-15

As shown in Table 6, the material-adding operations, procedures, analytical method and workup of Comparative Examples 3-15 are consistent with Example 58 except that the metallic catalysts changed. The experimental results are listed in Table 6. For ease of analysis, the results of Example 58 are also listed in Table 6.

TABLE 6

| | | Catalyst | Compound | Sulfoxide | Compound | Compound | Sulfoxide in |
|---|---|---|---|---|---|---|---|
| | | quantity | I area^{a} | area^{b} | II purity | II yield | Compound II |
| Example | Catalyst | (mol %) | (%) | (%) | (%) | (%) | (%) |
| Example 58 | Hexacarbonyltungsten | 0.5 | 0 | 0.18 | 99.2 | 98.6 | 0.02 |
| Comparative | Sodium | 0.5 | 0 | 5.4 | 94.1 | 93.5 | 5.1 |
| Example 3 | tungstate | | | | | | |
| Comparative | Molybdenum | 0.5 | 0 | 57.3 | / | / | / |
| Example 4 | carbide | | | | | | |
| Comparative | Molybdic | 0.5 | 0 | 46.7 | / | / | / |
| Example 5 | acid | | | | | | |
| Comparative | Sodium | 0.5 | 0 | 48.5 | / | / | / |
| Example 6 | molybdate | | | | | | |
| Comparative | Ammonium | 0.5 | 0 | 45.7 | / | / | / |
| Example 7 | molybdate | | | | | | |
| | tetrahydrate | | | | | | |
| Comparative | molybdenum | 0.5 | 0 | 55.3 | / | / | / |
| Example 8 | dioxide | | | | | | |
| Comparative | Phosphomolybdic | 0.5 | 0 | 53.1 | / | / | / |
| Example 9 | acid hydrate | | | | | | |
| Comparative | Silico | 0.5 | 0 | 45.3 | / | / | / |
| Example 10 | molybdic | | | | | | |
| | acid | | | | | | |
| Comparative | Tungsten | 0.5 | 0 | 11.3 | / | / | / |
| Example 11 | oxide | | | | | | |
| Comparative | Tungsten | 0.5 | 0 | 42.1 | / | / | / |
| Example 12 | chloride | | | | | | |
| Comparative | Tungsten | 0.5 | 0 | 5.8 | 93.4 | 92.6 | 5.6 |
| Example 13 | | | | | | | |
| Comparative | Tungsten | 0.5 | 0 | 12.5 | 86.7 | 86.0 | 12.3 |
| Example 14 | disulphide | | | | | | |
| Comparative | phosphotungstic | 0.5 | 0 | 7.4 | 91.7 | 91.2 | 7.2 |
| Example 15 | acid | | | | | | |

Note:
The “catalyst quantity (mol %)” in the table is the percentage of moles of the respective metal element in the catalyst to moles of Compound I.

The applicant declares that the present application illustrates the method of preparing the Compound II by the above embodiments, but the present application is not limited to the above embodiments, i.e., it does not mean that the present application must be relied on the above embodiments to be implemented. It should be clear to those skilled in the art that any improvement of the present application, equivalent substitution of each raw material of the product of the present application and addition of auxiliary ingredients, selection of specific ways, etc., all fall within the scope of protection and disclosure of the present application.

What is claimed is:

1. A method for preparing Compound II, comprising the following steps:
using Compound I as a raw material, performing a reaction under the action of an organometallic catalyst and a phase transfer catalyst, and oxidizing with hydrogen peroxide to obtain Compound II; and a reaction formula is as follows: 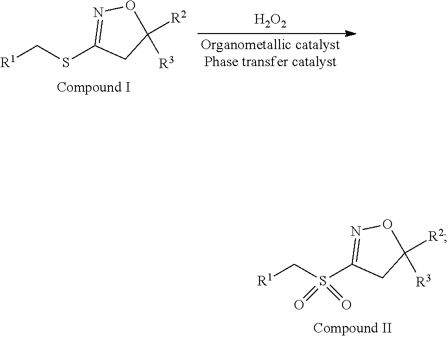
wherein R¹ is 1-methyl-3-trifluoromethyl-5-difluoromethoxy-1H-pyrazol-4-yl, and R² and R³ are each methyl;
the organometallic catalyst and Compound I has a molar ratio of (0.0001-0.018):1.

2. The method according to claim 1, wherein the organometallic catalyst is an organotungsten catalyst and an organomolybdenum catalyst.

3. The method according to claim 2, wherein the organotungsten catalyst comprises any one or a combination of at least two of hexacarbonyltungsten, tungsten (V) ethoxide, tungsten (VI) ethoxide, tungsten (VI) isopropoxide, bis(tert-butylimido)bis(dimethylamide)tungsten (VI), trimethylphenyltungsten tricarbonyl, bis(acetonitrile) tetracarbonyltungsten, (1,1-bis(diphenylphosphono)ferrocene) tetracarbonyltungsten, and the like organic tungsten-containing metal compounds, and further preferably hexacarbonyltungsten.

4. The method according to claim 2, wherein the organomolybdenum catalyst comprises any one or a combination of at least two of hexacarbonylmolybdenum, molybdenum acetate and polymers thereof, molybdenum iso-octoate, molybdenum di(ethylbenzene), molybdenum(V) isopropoxide, bis(t-butylimido)bis(dimethylamino)molybdenum(VI), tris(acetonitrile)molybdenum tricarbonyl, molybdenum(V) trichloroisopropoxide, (propylcyclopentadienyl)molybdenum(I) tricarbonyl dimer, (1,1′-bis(diphenylphosphor)ferrocene)tetracarbonylmolybdenum, and the like organic molybdenum-containing metal compounds, and further preferably hexacarbonylmolybdenum.

5. The method according to claim 1, wherein the organometallic catalyst and Compound I has a molar ratio of (0.003-0.018):1, preferably (0.005-0.015):1, more preferably 0.003:1, more preferably 0.004:1, more preferably 0.005:1, more preferably 0.008:1, more preferably 0.010:1, more preferably 0.012:1, and further more preferably 0.015:1.

6. The method according to claim 1, wherein the phase transfer catalyst is selected from any one or a combination of at least two of polyethers, crown ethers, tertiary amines, quaternary ammonium salts, quaternary ammonium bases or quaternary phosphonium salts, and further preferably quaternary ammonium salts.

7. The method according to claim 6, wherein the quaternary ammonium salt comprises any one or a combination of at least two of benzyltriethylammonium chloride, tetrabutylammonium bromide, tetrabutylammonium chloride, tetrabutylammonium hydrogen sulfate, trioctylmethylammonium chloride, dodecyltrimethylammonium chloride, tetradecyltrimethylammonium chloride, or tetramethylammonium chloride, and further preferably tetrabutylammonium bromide.

8. The method according to claim 1, wherein the phase transfer catalyst and Compound I has a molar ratio of (0.005-0.02):1, further preferably (0.005-0.018):1, and more preferably 0.01:1.

9. The method according to claim 1, wherein the reaction is carried out in the presence of an acid.

10. The method according to claim 9, wherein the acid is an organic acid, an inorganic acid, or a substance capable of reacting in situ with water to form an acid;
preferably, the organic acid is selected from any one or a combination of at least two of formic acid, acetic acid, difluoroacetic acid, trifluoroacetic acid, dichloroacetic acid, or trichloroacetic acid;
preferably, the inorganic acid is selected from any one or a combination of at least two of sulfuric acid, hydrochloric acid, nitric acid, phosphoric acid, boric acid, chlorosulfonic acid or chlorous sulfonic acid;
preferably, the substance capable of reacting in situ with water to form an acid is selected from any one or a combination of at least two of sulfonyl chlorides, thionyl chloride, acetyl chloride, dichloroacetyl chloride, trichloroacetyl chloride, trifluoroacetyl chloride, trifluoroacetyl fluoride, acetic anhydride, trifluoroacetic anhydride, peroxyacetic acid, triphosgene, chlorine, or bromine;

11. The method according to claim 10, wherein a molar ratio of the acid or the substance capable of reacting in situ with water to form an acid to Compound I is (0.001-1):1, preferably (0.01-0.3):1, and more preferably 0.1:1.

12. The method according to claim 1, wherein the hydrogen peroxide is aqueous hydrogen peroxide solution and has a concentration of 10-70%, preferably 20-50%, more preferably 30-50%, and further more preferably 50%.

13. The method according to claim 1, wherein the hydrogen peroxide and Compound I has a molar ratio of (2.0-6.0):1, preferably (2.05-4.0):1, more preferably (2.05-3.00):1, more preferably (2.0-2.1):1, and further more preferably 2.05:1.

14. The method according to claim 1, wherein the hydrogen peroxide is added to the reaction system in a dropwise manner;
preferably, the dropwise addition is conducted over a period of 0.5-3 h.

15. The method according to claim 1, wherein the reaction is carried out in an organic solvent, and the organic solvent is immiscible with water.

16. The method according to claim 15, wherein the organic solvent is selected from any one or a combination of at least two of aliphatic hydrocarbons, aromatic hydrocarbons, or halogenated aromatic hydrocarbons.

17. The method according to claim 15, wherein the organic solvent comprises any one or a combination of at least two of 1,2-dichloroethane, dichloromethane, chloroform, 1,1,2-trichloroethane, toluene, xylene, mesitylene, chlorobenzene, or dichlorobenzene, and further preferably 1,2-dichloroethane.

18. The method according to claim 1, wherein the reaction is carried out at a temperature of 30-90° C., preferably 40-60° C., and more preferably 50-60° C.;
preferably, the reaction is carried out for a period of 2.5-12 h.

19. The method according to claim 1, wherein the method further comprises a post-treatment step after the reaction;
wherein the post-treatment step comprises: adding water to recover the solvent by atmospheric distillation, adding methanol and stirring for crystallization, filtering, water washing, and drying to obtain Compound II.

20. A product prepared by the preparation method according to claim 1, wherein the method comprises the following steps:
adding hydrogen peroxide dropwise to an organic solvent system containing Compound I, an organometallic catalyst, and a phase transfer catalyst, reacting at 30-90° C. for 2.5-12 hours, adding water and performing atmospheric pressure distillation to recover the solvent, adding methanol and stirring for crystallization, filtering, water washing, and drying to obtain Compound II;
wherein the organometallic catalyst is an organotungsten catalyst and organomolybdenum catalyst, and the organometallic catalyst and Compound I has a molar ratio of (0.0001-0.018):1;
the phase transfer catalyst is selected from any one or a combination of at least two of polyethers, crown ethers, tertiary amines, quaternary ammonium salts, quaternary ammonium hydroxides, or quaternary phosphonium salts, and the phase transfer catalyst and Compound I has a molar ratio of (0.005-0.02):1;
the hydrogen peroxide is aqueous hydrogen peroxide solution and has a concentration of 10-70%, and hydrogen peroxide and Compound I has a molar ratio of (2.0-6.0):1;
the organic solvent is selected from any one or a combination of at least two of halogenated aliphatic hydrocarbons, aromatic hydrocarbons, or halogenated aromatic hydrocarbons.
